(19) United States
(12) Patent Application Publication
(10) Pub. No.: US 2010/0191162 A1
(43) Pub. Date: Jul. 29, 2010

(54) APPENDAGE SPLINT

(75) Inventors: Shin-Ju D. CHANG, South Pasadena, CA (US); Christine Y. Chang, South Pasadena, CA (US)

Correspondence Address: Don B. Finkelstein, Esq.;LAW OFFICES OF DON FINKELSTEIN, Suite 216, 3858 Carson Street, Torrance, CA, 90503, US

(73) Assignee: ELITE ORTHOPAEDICS, INC., South El Monte, CA (US)

(21) Appl. No.: 12/362,434

(22) Filed: Jan. 29, 2009

(51) Int. Cl. A61F 5/00

(52) U.S. Cl. 602/5

(57) ABSTRACT

In an embodiment, an appendage splint is disclosed. The appendage splint may include or comprise a support member including a lateral member configured to wrap and couple with a portion of the support member.

### TECHNICAL FIELD

The present technology relates to the field of appendage splints.

### BACKGROUND

It is to be understood that various limbs or physical members may be associated with one or more appendages coupled therewith, wherein these appendages are capable of moving relative to such limbs or physical members. For example, a body may include a foot that is moveably coupled with a leg such that the foot is able to move relative to the leg. Similarly, a body may include a lower arm that is moveably coupled with an upper arm such that the lower arm is able to move relative to the upper arm.

In certain situations, it may be beneficial to hinder a degree of movement of an appendage relative to a limb or physical member with which the appendage is coupled. For example, in the event that a person's foot or ankle becomes damaged, it may be beneficial to the healing process if the amount of movement of such foot relative to the leg with which such foot is coupled is minimized so as to speed the recovery of the suffered injury as well as prevent further damage.

### SUMMARY

This Summary is provided to introduce a selection of concepts in a simplified form that are further described below in the Detailed Description. This Summary is not intended to identify key or essential features of the claimed subject matter, nor is it intended to be used as a limitation in determining the scope of the claimed subject matter.

In an embodiment, an appendage splint is disclosed. The appendage splint may include or comprise a support member including a lateral member configured to wrap and couple with a portion of the support member.

Additionally, in one embodiment, an appendage splint is disclosed, wherein the appendage splint may include or comprise a support member, and wherein the support member may include or comprise a lateral member configured to move from a first position to a second position relative to the support member and couple with the support member when the lateral member is positioned in the second position.

Moreover, in an embodiment, an appendage splint is disclosed, wherein the appendage splint may include or comprise a support member that may include or comprise a lateral extension extending from the support member. The appendage splint may further include or comprise a lateral member extending from the lateral extension, wherein the lateral member may include or comprise a base member configured to move from a first position to a second position relative to the support member, and wherein the lateral member may further include or comprise a connecting member extending from the base member, wherein the connecting member is configured to connect with the support member when the base member is positioned in the second position.

Furthermore, in one embodiment, an appendage splint is disclosed, wherein the appendage splint may include or comprise a support member that may include or comprise a lateral extension extending from the support member. The appendage splint may further include or comprise a lateral member extending from the lateral extension, wherein the lateral member may include or comprise a rigid base member configured to move from a first position to a second position relative to the support member, and wherein the lateral member may further include or comprise a coupling member extending from the rigid base member, wherein the coupling member is configured to couple with the support member when the rigid base member is positioned in the second position.

### DESCRIPTION OF THE DRAWINGS

The accompanying drawings, which are incorporated in and form a part of this specification, illustrate embodiments of the present technology, and, together with the Detailed Description, serve to explain principles discussed below.

FIG. 1A is an anterior view of an exemplary configuration of an appendage splint in accordance with an embodiment.

FIG. 1B is a posterior view of an exemplary configuration of an appendage splint in accordance with an embodiment.

FIGS. 2A and 2B are lateral views of an exemplary implementation of an appendage splint in accordance with an embodiment.

FIG. 2C is a plantar view of an exemplary implementation of an appendage splint in accordance with an embodiment.

FIGS. 3A, 3B and 3C are lateral views of an exemplary implementation of an appendage splint in accordance with an embodiment.

The drawings referred to in this description are not to be understood as being drawn to scale except if specifically noted. Additionally, it is noted that these drawings are exemplary in nature, and that the spirit and scope of the present technology is not limited to the exemplary embodiments presented in such drawings.

### DETAILED DESCRIPTION

Reference will now be made in detail to embodiments of the present technology, examples of which are illustrated in the accompanying drawings. While the present technology will be described in conjunction with various embodiments, these embodiments are not intended to limit the present technology. Rather, the present technology is to be understood as encompassing obvious alternatives, modifications and equivalents, which may be included within the spirit and scope of the various embodiments as defined by the appended claims. Indeed, various exemplary embodiments, or aspects thereof, may be combined in accordance with the spirit and scope of the present technology.

Furthermore, in the following Detailed Description numerous specific details are set forth in order to provide a thorough understanding of the present technology. However, the present technology may be practiced without these specific details. In other instances, well-known methods, procedures, and components have not been described in detail so as to not unnecessarily obscure aspects of the exemplary embodiments presented herein.

Moreover, for purposes of the following discussion, the term “plurality” refers to two or more. Additionally, the terms “primary” and “secondary”, as well as the terms “first”, “second” and “third”, are not to be construed as imparting a numerical order or a degree of importance.

The foregoing notwithstanding, for purposes of the following discussion, the term “anterior” refers to a front view, portion, section, region, etc., while the term “posterior” refers to a back or rear view, portion, section, region, etc. Similarly, the term “plantar” refers to a bottom view, portion, section, region, etc., while the term “dorsal” refers to a top view, portion, section, region, etc. In addition, the term “medial” refers to a middle view, portion, section, region, etc., while the term “lateral” refers to a side view, or a portion, section, region, etc., positioned away from a medial portion.

Furthermore, for purposes of the following discussion, the terms “dorsiflexion” and “plantarflexion” refer to an upward and downward movement, respectively, of an appendage relative to a limb, wherein the appendage is moveably coupled with such limb. The terms “abduction” and “adduction” refer to the movement of an appendage away from and towards, respectively, a median axis of a body with which the appendage is associated. The terms “inversion” and “eversion” refer to a twisting movement of an appendage inward and outward, respectively, wherein the twisting movement is relative to a limb or body with which the appendage is coupled or associated. Moreover, the terms “supination” and “pronation” refer to tri-planar motions of an appendage towards and away from, respectively, a median axis of a body with which the appendage is associated.

### Overview

In an embodiment, an appendage splint is disclosed, wherein the appendage splint is configured to support an appendage relative to a limb with which the appendage is moveably coupled such that a degree of movement of the appendage relative to the limb is hindered or precluded. Consider the example where a person's foot or ankle is injured. It may be beneficial to the healing process of the injured member if the foot is supported relative to the leg with which the foot is moveably coupled. Specifically, the injured member may heal more quickly or more effectively if the amount of movement of the person's foot relative to the leg with which such foot is moveably coupled is minimized.

Thus, an embodiment provides a means of supporting an injured member or appendage relative to a limb or body with which the injured member or appendage is moveably coupled, such as to expedite or aid the healing process of the suffered injury. Indeed, an appendage splint in accordance with the present technology may be implemented as a dorsal night splint that is capable of being more easily applied to and removed from a patient's leg, such as during periods of bed rest when the patient is being treated for adverse appendage conditions (e.g., plantar fasciitis), or when ankle immobilization is desired.

Various different configurations of an appendage splint may be implemented within the spirit and scope of the present technology. Pursuant to one exemplary configuration, an appendage splint includes a support member that has a rigid section configured to be held adjacent to an anterior portion of a limb. The appendage splint further includes a lateral member that is moveably coupled with a first portion of the support member such that the lateral member is configured to wrap around an appendage associated with the aforementioned limb. Furthermore, the lateral member includes a coupling member configured to couple with a second portion of the support member when the lateral member is wrapped around the appendage, wherein the first and second portions of the support member may be contained within or associated with different lateral sides of the support member, such that the lateral member is coupled with different portions of the support member, and such that the appendage is supported relative to the aforementioned limb.

Additionally, pursuant to one embodiment, a portion of the lateral member is substantially rigid, and this rigid portion is configured to be positioned adjacent to a plantar portion of an appendage when the lateral member is wrapped around the appendage. Due to a degree of rigidity of this portion of the lateral member, as well as to an ability of the lateral member to be wrapped taut around the appendage, a degree of movement of such appendage relative to a limb with which the appendage is moveably coupled is hindered to a greater degree than would be achievable if the portion of the lateral member contacting the plantar portion of the appendage were configured to be substantially flexible or non-rigid.

Thus, in accordance with an embodiment, an appendage splint includes a support member, and a base member coupled with a portion of the support member, wherein the base member is configured to move from a first position to a second position relative to the support member. The appendage splint further includes a coupling member coupled with the base member, wherein the coupling member is configured to couple with a different portion of the support member when the base member is positioned in the second position such that a degree of support is offered. This degree of support may be offered based on a degree of rigidity associated with the base member, and a degree of movement may be hindered in response to the degree of support being utilized.

The foregoing notwithstanding, it is further noted that the spirit and scope of the present technology is not limited to the aforementioned components of an appendage splint. For example, in an embodiment, a wrapping element is associated with the support member, wherein the wrapping element is configured to hold the support member in place so as to increase the degree of support that is offered. Moreover, pursuant to one embodiment, the support member includes a plurality of lateral extensions configured to flex so as to increase the offered degree of support. Furthermore, in accordance with one exemplary implementation, a force absorbing element is associated with the support member, wherein the force absorbing element is configured to absorb a force associated with the support member being held in place such that a level of comfort associated with the support member being held in place is increased.

Additionally, in an embodiment, the appendage splint includes a primary hinge area coupled to the support member and the base member, wherein the primary hinge area is configured to enable the base member to move relative to the support member. The appendage splint further includes a secondary hinge area coupled to the base member and the coupling member, wherein the secondary hinge area is configured to enable the coupling member to move relative to the base member. Indeed, in accordance with one exemplary configuration, the support member, the primary hinge area, the base member, the secondary hinge area, and the coupling member include different sections of a single splint member having varying degrees of thickness, wherein different degrees of thickness are associated with the different sections, and wherein degrees of rigidity associated with the support member and the base member are greater than different degrees of rigidity associated with the primary hinge area and the secondary hinge area.

Moreover, as noted above, an embodiment provides that an appendage splint includes a coupling member coupled with a base member, wherein the coupling member is configured to couple with a portion of the support member when the base member is positioned in the aforementioned second position such that a degree of support is offered. It is noted that, in one embodiment, the coupling member includes a coupler configured to couple the coupling member to the support member such that the aforementioned degree of support may be offered.

Indeed, in accordance with one configuration, an outer layer of material (e.g., an outer liner) is wrapped around or associated with the support member, wherein at least a portion of the outer layer is positioned adjacent to the portion of the support member with which the coupling member is configured to couple. Moreover, the coupling member includes a coupler configured to couple to this portion of the outer layer so as to couple the coupling member with the support member such that the aforementioned degree of support may be offered.

The foregoing notwithstanding, the present technology is not limited to these exemplary embodiments. Indeed, it is noted that various different configurations of an appendage splint may be implemented within the spirit and scope of the present technology.

### Exemplary Configurations and Implementations

Various exemplary configurations and implementations for an appendage splint will now be described. However, the present technology is not limited to these exemplary configurations and implementations. Indeed, other configurations and implementations may be utilized within the spirit and scope of the present technology.

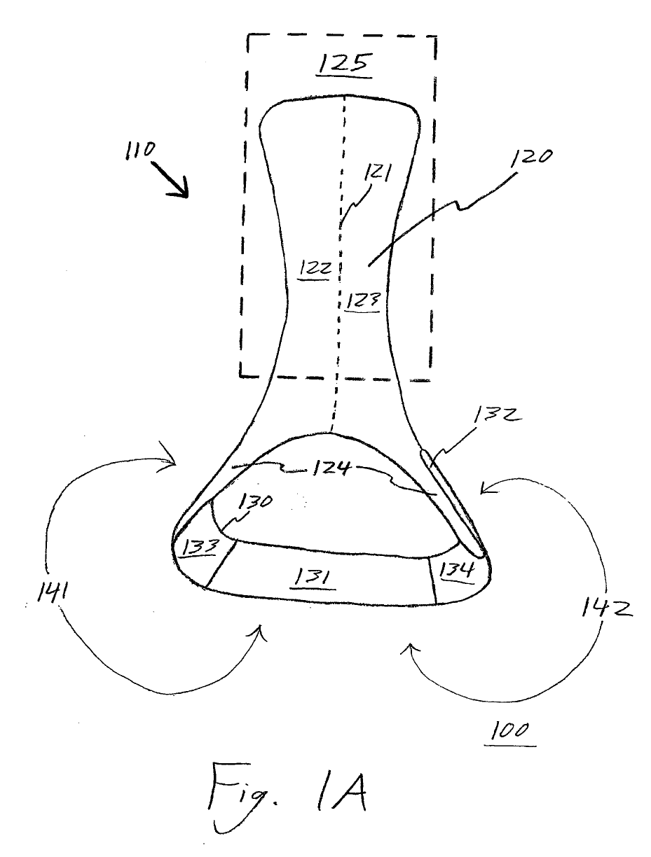

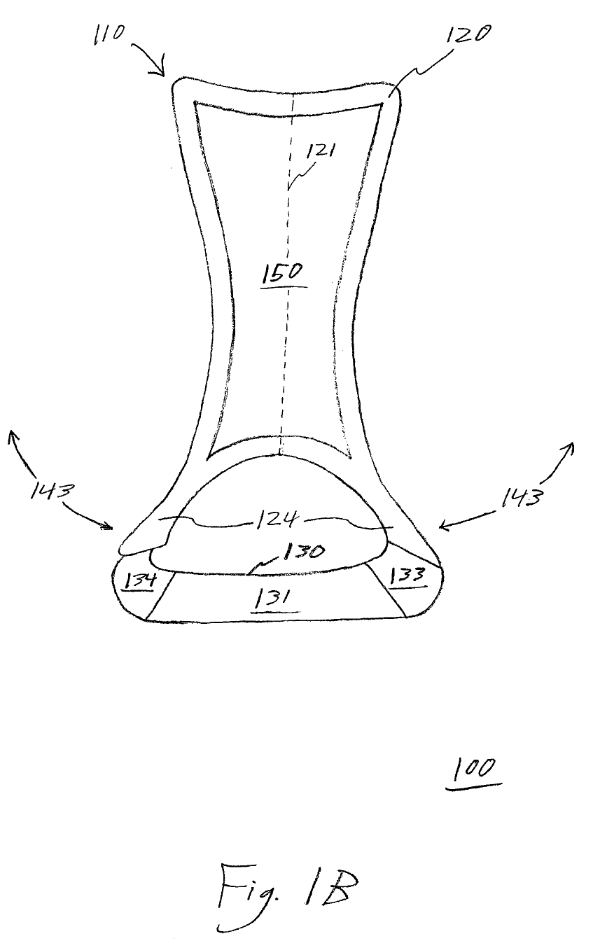

With reference now to FIG. 1A, an anterior view of an exemplary configuration 100 of an appendage splint 110 in accordance with an embodiment is shown. Appendage splint 110 includes a support member 120 having a major axis 121 along a medial length of support member 120. Major axis 121 separates support member 120 into a first lateral side 122 and a second lateral side 123. Furthermore, appendage splint 110 also includes a lateral member 130 moveably coupled with a portion of support member 120, and more specifically with first lateral side 122, wherein lateral member 130 includes a coupling member 132 that is configured to couple lateral member 130 with a different portion of support member 120 such that lateral member 130 becomes coupled with multiple portions of support member 120, and such that a degree of movement of lateral member 130 relative to support member 120 is diminished in response to lateral member 130 being coupled with multiple portions of support member 120.

To further illustrate, and with reference still to the embodiment shown in FIG. 1A, appendage splint 110 includes a plurality of lateral extensions 124 that extend away from a top portion 125 of support member 120. For example, lateral extensions 124 extend away from generally opposed sides of support member 120, which are divided by major axis 121. Furthermore, lateral extensions 124 are configured to adhere around an appendage that is positioned between lateral extensions 124 such that lateral extensions 124 conform to a shape of the appendage.

Thus, an embodiment provides that appendage splint 110 includes support member 120, which includes generally opposite sides divided by major axis 121, as well as lateral extensions 124, which are configured to adhere around an appendage positioned between lateral extensions 124. Pursuant to one embodiment, lateral member 130 is moveably coupled to a lateral extension from among lateral extensions 124. Furthermore, lateral member 130 is configured to wrap around an appendage positioned between lateral extensions 124 such that coupling member 132 is able to couple with support member 120, such as by means of an adhesive member associated with coupling member 132.

Moreover, in accordance with an embodiment, the portion of support member 120 with which lateral member 130 is moveably coupled is located within a first side from among the generally opposite sides of support member 120 (e.g., first lateral side 122), and coupling member 132 is configured to couple with a different portion of support member 120, which is located within a different side from among the aforementioned generally opposite sides (e.g., second lateral side 123). Alternatively, an example provides that lateral member 130 is elongated such that lateral member 130 is able to wrap around an appendage positioned between lateral extensions 124, and such that coupling member is able to be positioned adjacent to and coupled with first lateral side 122. Thus, it is noted that the present technology is not limited to coupling member 132 being configured so as to couple coupled with a specific portion or component of appendage splint 110, such as a specific lateral side or dorsal section of support member 120.

Indeed, in an embodiment, appendage splint 110 is configured such that coupling member 132 may be coupled with different portions of appendage splint 110 depending on a positioning of coupling member 132 relative to support member 120. Consider the example where multiple adhesion receptacles are coupled or affixed to different locations along support member 120, and where coupling member 132 is configured to couple with or adhere to each of these adhesion receptacles, although not simultaneously. Rather, coupling member 132 is positioned adjacent to one of these adhesion receptacles based on a positioning of lateral member 130 relative to support member 120.

Furthermore, one exemplary configuration provides that an outer surface of support member 120 is configured to couple with coupling member 132 such that coupling member 132 is able to couple with different portions of this outer surface based on a positioning of lateral member 130 relative to support member 120. Alternatively, pursuant to one example, support member 120 is wrapped in an adhesion receptive material that is configured to couple with coupling member 132 such that coupling member 132 is able to couple with different portions of support member 120 by bonding with different sections of the adhesion receptive material.

With reference still to FIG. 1A, lateral member 130 includes a base member 131 configured to move relative to support member 120, such as in one or more of exemplary directions of rotation 141. Lateral member 130 further includes coupling member 132, which is configured to move relative to base member 131, such as in one or more of exemplary rotational directions 142. In this manner, base member 131 is moveably coupled with support member 120, and coupling member 132 is moveably coupled with base member 131.

To further illustrate, in an embodiment, appendage splint 110 includes support member 120, which includes a lateral extension that extends away from top portion 125 of support member 120. Appendage splint 110 further includes lateral member 130, which is associated with support member 120 such that a first portion of lateral member 130 is coupled with the lateral extension of support member 120. Additionally, lateral member 130 is configured to move relative to support member 120 such that a second portion of lateral member 130 is able to couple with support member 120, and such that a degree of support is offered in response to the second portion of lateral member 130 being coupled with support member 120.

With reference still to the previous embodiment, in accordance with one configuration, lateral member 130 includes base member 131, which is coupled with support member 120. Additionally, the aforementioned degree of support is offered based on a degree of rigidity associated with base member 131. Furthermore, an embodiment provides that the lateral extension of support member 120, with which lateral member 130 is coupled, is configured to flex so as to increase the degree of support when lateral member 130 is coupled to multiple portions of support member 120.

It is noted that coupling member 132 may be configured to couple with different portions of appendage splint 110. For example, coupling member 132 may be configured to couple with a lateral or dorsal section of support member 120 depending on, for example, the size of the appendage that lateral member 130 is wrapped around, as well as the overall length of lateral member 130. In one embodiment, however, base member 131 is configured to be positioned adjacent to a plantar portion of an appendage, and lateral member 130 is elongated to a degree that enables lateral member 130 to wrap around a dorsal section of support member 120 such that coupling member 132, which extends from base member 131, is displaced around the dorsal section of support member 120 and then adjacent to a plantar surface of base member 131 such that coupling member 132 is able to couple with the plantar surface.

In an embodiment, lateral member 130 also includes a primary hinge area 133 coupled to or between support member 120 and base member 131, wherein primary hinge area 133 is configured to enable base member 131 to move relative to support member 120. Lateral member 130 further includes a secondary hinge area 134 coupled to or between base member 131 and coupling member 132, wherein secondary hinge area 134 is configured to enable coupling member 132 to move relative to base member 131. Moreover, in accordance with an exemplary configuration, support member 120 and base member 131 are relatively rigid in comparison to primary hinge area 133 and secondary hinge area 134. This relatively rigid nature of support member 120 and base member 131 aids appendage splint 110 to support an appendage relative to a limb with which the appendage is moveably coupled, such as further discussed herein.

It is noted that various type of hinges may be implemented within the spirit and scope of the present technology. For example, primary hinge area 133 and/or secondary hinge area 134 may include hinge elements that are attached to portions of support member 120, such as to lateral extensions 124, after support member 120 has been fabricated. Pursuant to one embodiment, however, primary hinge area 133 and/or secondary hinge area 134 include flexible portions of the same piece of material used to fabricate support member 120. Indeed, in accordance with an exemplary implementation, a single piece of material includes support member 120 as well as lateral member 130, including its constituent components. This piece of material may include, for example, a plastic or other material capable of being molded into the form of an appendage splint or shell, such as during a mold injection process, wherein the thicker portions of the material are relatively rigid while thinner portions of the material are relatively flexible in response to an applied force.

To illustrate, in an embodiment, support member 120, primary hinge area 133, base member 131, secondary hinge area 134, and coupling member 132 include different sections of a single splint member having varying degrees of thickness, wherein different degrees of thickness are associated with these different sections. In particular, this single splint member is fabricated such that support member 120 and base member 131 are relatively rigid in comparison to primary hinge area 133 and secondary hinge area 134, and such that primary hinge area 133 and secondary hinge area 134 are relatively flexible in comparison to support member 120 and base member 131. In this manner, an exemplary configuration provides that the fabricated splint member includes thinner portions and thicker portions, wherein the relative levels of thicknesses associated with these different portions, along with the general shape of the fabricated unit, define the constituent components of appendage splint 110.

To further illustrate, and in accordance with an example, in so much as significant portions of both support member 120 and base member 131 are thicker than primary hinge area 133 and secondary hinge area 134, degrees of rigidity associated with support member 120 and base member 131 are greater than different degrees of rigidity associated with primary hinge area 133 and secondary hinge area 134. This causes primary hinge area 133 and secondary hinge area 134 to be relatively flexible in comparison to support member 120 and base member 131. Moreover, it is noted that primary hinge area 133 is configured to enable base member 131 to move relative to support member 120 based on a degree of flexibility associated with primary hinge area 133 being greater than another degree of flexibility associated with support member 120. Similarly, secondary hinge area 134 is configured to enable coupling member 132 to move relative to base member 131 based on a degree of flexibility associated with secondary hinge area 134 being greater than another degree of flexibility associated with base member 131.

Thus, an embodiment provides that degrees of thickness associated with support member 120 and base member 131 are greater than different degrees of thickness associated with primary hinge area 133 and secondary hinge area 134 such that support member 120 and base member 131 are substantially rigid in comparison to primary hinge area 133 and secondary hinge area 134, and such that primary hinge area 133 and secondary hinge area 134 are substantially flexible in comparison to support member 120 and base member 131. However, the spirit and scope of the present technology is not limited to the implementation of a single splint member, such as discussed above. Indeed, an embodiment provides that appendage splint 110 includes different pieces of material that are coupled together.

Moreover, as noted above, an exemplary configuration provides that appendage splint 110, or a component thereof, is made of or includes a plastic material. However, the spirit and scope of the present technology is not limited to the use of a plastic material.

For example, one or more components of appendage splint 110 may be made of or include a metallic material. To illustrate, an embodiment provides that support member 120 and/or base member 131 are constructed from one or more molded pieces of a metallic substrate configured to be substantially rigid such that support member 120 and base member 131 are capable of providing a degree of support.

Additionally, in accordance with an example, a hinge area, such as primary hinge area 133 and/or secondary hinge area 134, may be constructed of one or more metallic hinge components. Consider the example where a hinge device is implemented includes two metal hinge components that are moveably coupled together. One of the hinge components is coupled with support member 120 while the other hinge component is coupled with base member 131 such that base member 131 is able to move relative to support member 120. In this manner, primary hinge area 133 includes a metal hinge device. Indeed, it is noted that secondary hinge area 134 may also include a metal hinge device, such as described above.

Moreover, in an embodiment, one or more components of appendage splint 110, such as primary hinge area 133 and secondary hinge area 134, may be made of or include a material or fabric capable of being stretched. Consider the example where primary hinge area 133 and secondary hinge area 134 are constructed from a stretchable material or fabric. Primary hinge area 133 and secondary hinge area 134 are able to flex such that base member 131 and coupling member 132 may be repositioned relative to support member 120. However, in so much as primary hinge area 133 and secondary hinge area 134 are capable of being stretched, lateral member 130 may be wrapped tighter around appendage 220 than would be achievable if primary hinge area 133 and secondary hinge area 134 were not stretchable.

Furthermore, in one embodiment, one or more components of appendage splint 110 may be inflatable, such as to increase a degree of comfort associated with appendage splint 110. Consider the example where support member 120 includes or is coupled with an inflatable chamber. When this chamber is inflated, a degree of comfort associated with support member 120 being held adjacent to limb 210 is increased.

Thus, it is noted that appendage splint 110, or one or more components thereof, may be made of a metal or plastic material, or a stretchable fabric. In one embodiment, a combination of materials are utilized to fabricate appendage splint 110, such as by constructing support member 120 and base member 131 from a rigid metal or plastic material, while primary hinge area 133 and secondary hinge area 134 include a stretchable fabric material. Moreover, one or more components of appendage splint 110 may include or be coupled with an inflatable member, such as described above. However, as previously stated, the spirit and scope of the present technology is not limited to the implementation of any particular material.

With reference still to FIG. 1A, in accordance with an embodiment, coupling member 132, which may be either rigid or flexible, is further configured to couple with support member 120 such that base member 131 is substantially immobilized with respect to support member 120. Consider the example where coupling member 132 couples with second lateral side 123, as shown in FIG. 1A. In so much as lateral member 130 is coupled with first lateral side 122, and in so much as base member 131 is coupled with second lateral side 123 by means of coupling member 132, an embodiment provides that a degree of movement of base member 131 relative to support member 120 is substantially diminished in response to coupling member 132 being coupled with second lateral side 123. In accordance with an exemplary implementation, this immobilization of base member 131, along with a degree of rigidity associated with base member 131, aids appendage splint 110 to support an appendage relative to a limb with which the appendage is moveably coupled, such as further discussed herein.

With reference now to FIG. 1B, a posterior view of exemplary configuration 100 in accordance with an embodiment is shown. Support member 120 includes a plurality of flexible portions configured to move relative to major axis 121, such as in exemplary directions 143. Consider the example where lateral extensions 124 include or correspond to these flexible portions. Lateral extensions 124 are able to flex toward and/or away from one another such that a distance between lateral extensions 124 is adjustable. In this manner, the positioning of the aforementioned flexible portions of appendage splint 110 may be adjusted based on a distance between these flexible portions that comports with an implementation of exemplary configuration 100, such as to increase an efficacy of exemplary configuration 100 with regard to an objective thereof.

Indeed, in accordance with one exemplary implementation, lateral extensions 124 are configured to flex toward an appendage positioned between lateral extensions 124 such that lateral extensions 124 contact an outer surface of the appendage as well as mold to a shape of such outer surface. Moreover, it is noted that lateral member 130 is configured to wrap around the appendage, such as described above. However, in so much as lateral extensions 124 are molded to a shape of the outer surface of the appendage, the ability of lateral member 130 to be tightly drawn or wrapped around the appendage is increased. In this manner, an embodiment provides that lateral member 130 is configured to be wrapped taut around such an appendage, based on a degree of appendage size conformation associated with appendage splint 110, such that an ability of appendage splint 110 to support the appendage relative to a limb with which the appendage is moveably coupled is increased.

Moreover, in an embodiment, appendage splint 110 includes a force absorbing element 150 coupled with support member 120. Force absorbing element 150 is configured to absorb a force associated with support member 120 being held adjacent to a limb such that a level of comfort associated with support member 120 being held adjacent to such limb is increased. For example, force absorbing element 150 may include a relatively soft material, or an inflatable member, configured to mold to a portion of the limb that contacts force absorbing element 150. Additionally, the material is further configured to absorb an amount of mechanical force being exerted toward the limb such that the amount of force that the limb experiences, as well as a level of discomfort associated with such force, is diminished.

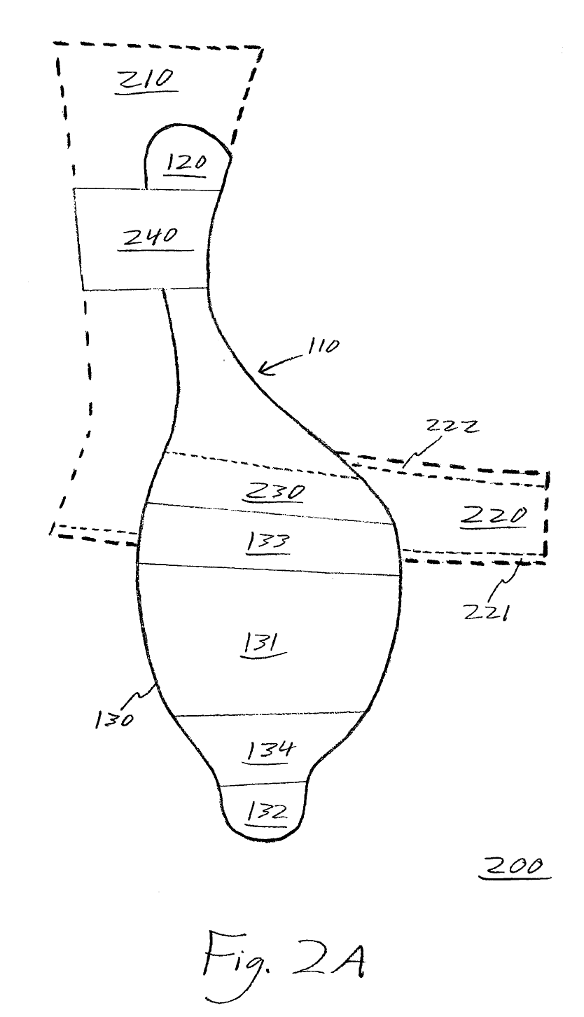

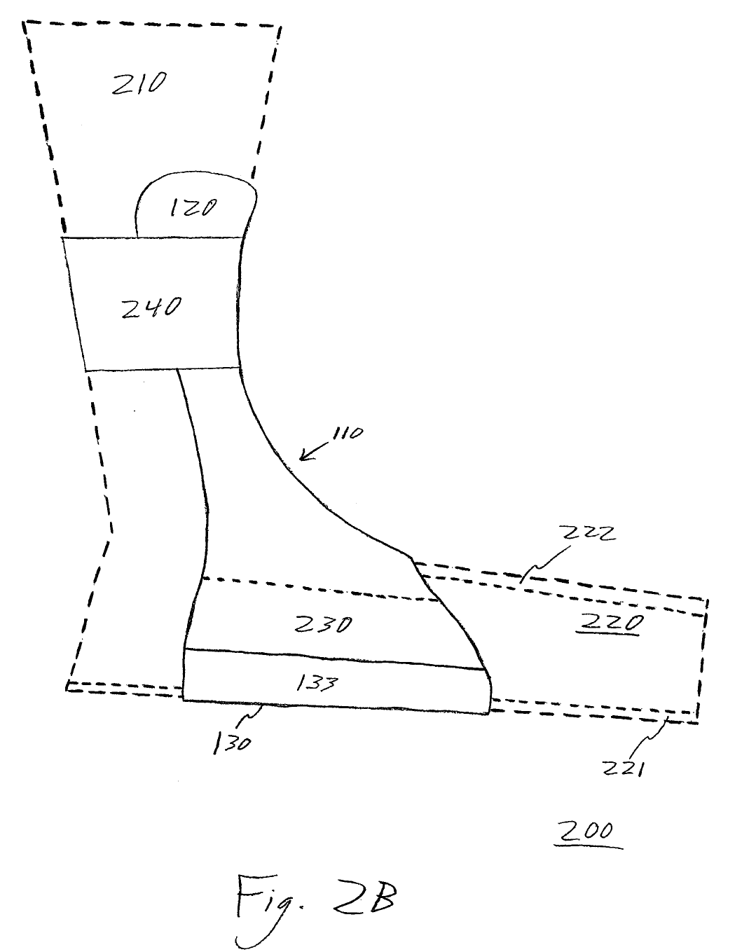

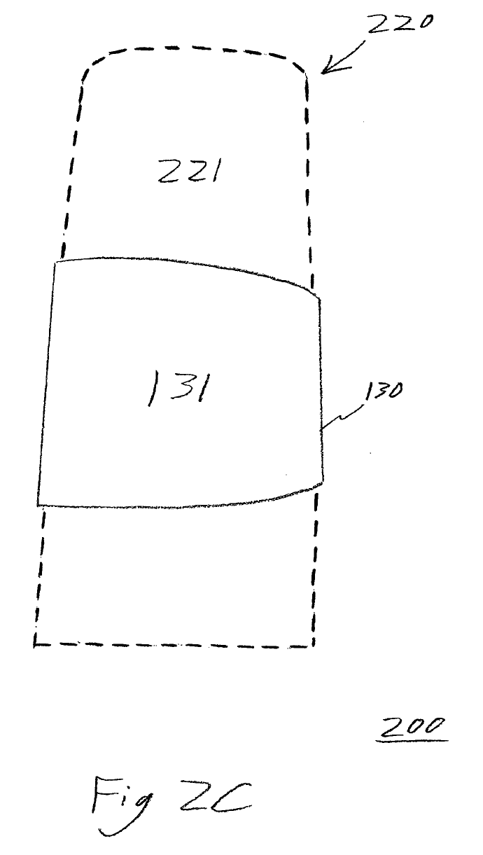

With reference now to FIG. 2A, a first lateral view of a first exemplary implementation 200 of appendage splint 110 in accordance with an embodiment is shown. In particular, appendage splint 110 is shown to include support member 120, which is configured to be positioned adjacent to a limb 210 (e.g., a lower leg), and lateral member 130, which is moveably coupled with a portion (e.g., a lateral portion) of support member 120, such as described above. In particular, lateral member 130 is configured to wrap around an appendage 220 (e.g., a foot) of limb 210, wherein appendage 220 includes a plantar portion 221 that is located substantially opposite to a dorsal portion 222 of appendage 220 along a length of appendage 220.

With reference still to FIG. 2A, lateral member 130 includes base member 131, which is configured to move relative to support member 120 so as to be positioned adjacent to a portion of an appendage 220 associated with limb 210, such as plantar portion 221 of appendage 220. Lateral member 130 further includes coupling member 132, which is coupled with base member 131 by means of secondary hinge area 134, such as described above, wherein coupling member 132 is configured to couple base member 131 with a portion of support member 120, such as a portion of support member 120 that is different than the portion of support member with which lateral member 130 is coupled, and such that base member 131 is configured to support appendage 220 relative to limb 210.

In accordance with one embodiment, support member 120 includes one or more flexible portions, such as flexible portion 230, which are configured to move, flex, or be positioned toward appendage 220. Indeed, in one example, these one or more flexible portions are configured to be positioned toward appendage 220 so as to contact appendage 220. In this manner, an ability of support member 120 to mold to appendage 220 is increased.

To further illustrate, consider the example where a lateral extension from among lateral extensions 124, as shown in FIGS. 1A and 1B, includes or corresponds to flexible portion 230. Flexible portion 230 is configured to flex toward appendage 220 when appendage 220 is positioned between lateral extensions 124 such that flexible portion 230 contacts an outer surface of appendage 220. Moreover, flexible portion 230 is further configured to mold to a shape of the outer surface of appendage 220, with which flexible portion 230 comes into contact, based on a degree of flexibility associated with flexible portion 230.

With reference still to FIG. 2A, in an embodiment, appendage splint 110 includes or is integrated with a wrapping element 240 (e.g., a calf strap), which is configured to wrap around limb 210 so as to hold support member 120 adjacent to limb 210. Consider the example where wrapping element 240 is configured to be wrapped taut around both support member 120 and limb 210 so as to hold support member 120 adjacent to limb 210. In so much as wrapping element is wrapped tightly around both support member 120 and limb 210, appendage splint 110 is held securely to limb 210.

Moreover, in an embodiment, wrapping element 240 is coupled with support member 120 by means of a coupling device. Consider the example where appendage splint 110 is wrapped in a fabric that substantially conforms to a shape of appendage splint 110. A coupling device is fastened to wrapping element 240, wherein the coupling device includes a plurality of small hooks configured to grasp portions of the fabric. This coupling device is then coupled with the fabric wrapped around appendage splint 110 such that the aforementioned hooks grasp the fabric, and such that wrapping element 240 is coupled with support member 120.

Thus, an embodiment provides that appendage splint 110 includes a number of different components, such as described above. Pursuant to one exemplary configuration, these components are configured to be self-aligned based on the manner in which such components are coupled or integrated together. Indeed, in accordance with one implementation, lateral member 130 is configured to wrap around an appendage such that coupling member 132 is positioned adjacent to a specific portion of support member 120 with which coupling member 132 is configured to couple. In this manner, the ease of operation associated with appendage splint 110 may be optimized.

With reference now to FIGS. 2B and 2C, a second lateral view and a plantar view of first exemplary implementation 200 in accordance with an embodiment are shown, respectively. In the illustrated embodiment, base member 131, which is hidden from view in FIG. 2B, but which is viewable from the plantar perspective shown in FIG. 2C, has been positioned adjacent to plantar portion 221 of appendage 220. In particular, base member 131 has rotated underneath appendage 220 such that base member 131 is in contact with plantar portion 221. Furthermore, coupling member 132, which is hidden from view in both figures, has been positioned adjacent to support member 120, and has been coupled with a portion of support member 120 with which coupling member has come into contact, such as described above.

Thus, in accordance with an embodiment, lateral member 130 is wrapped around appendage 220 in response to base member 131 rotating under plantar portion 221 and coupling member rotating relative to base member 131 and toward support member 120, such as described above. Furthermore, in so much as lateral member 130 has been wrapped around appendage 220, a degree of mobility of appendage 220 relative to limb 210 is hindered by appendage splint 110 such that appendage 220 is supported relative to limb 210.

The foregoing notwithstanding, it is noted that coupling member 132 may be coupled with different portions of support member 120 within the spirit and scope of the present technology. In one embodiment, however, the portion of support member 120 with which coupling member 132 is coupled is not located adjacent to plantar portion 221 of appendage 220. Therefore, it is noted that an embodiment provides that base member 131, and not coupling member 132, is located beneath appendage 220 when coupling member 132 has been coupled with support member 120. Furthermore, in so much as coupling member 132 is not located adjacent to plantar portion 221 when coupled with support member 120, a degree of support achievable with appendage splint 110 is increased due to a degree of rigidity associated with base member 131.

With reference still to FIG. 2B, in an embodiment, appendage splint 110 includes or is integrated with a wrapping element 240, such as described above. Pursuant to one exemplary implementation, wrapping element 240 is configured to wrap around limb 210 so as to hold support member 120 adjacent to limb 210, and so as to enable support member 120 to support limb 210 relative to appendage 220. To illustrate, consider the example where appendage splint 110 is held adjacent to limb 210 by means of wrapping element 240, and where appendage splint 110 is also attached to appendage 220 by means of lateral member 130 being wrapped around appendage 220, and more specifically, by means of coupling member 132 (not shown), which is coupled with support member 120. Thus, appendage splint 110 is attached to both limb 210 and appendage 220, which is moveably coupled with limb 210. However, in so much as a degree of rigidity is associated with one or more components of appendage splint 110, such as discussed herein, a degree of mobility of appendage 220 relative to limb 210 is hindered such that appendage 220 is supported by appendage splint 110 relative to limb 210.

Thus, with reference again to FIGS. 2A-2C, an embodiment provides that appendage splint 110 includes support member 120, as well as lateral member 130, which is coupled to support member 120. Moreover, lateral member 130 includes primary hinge area 133 and base member 131, wherein primary hinge area 133 is moveably coupled to support member 120, and wherein base member 131 is coupled to primary hinge area 133 such that base member 131 is configured to move relative to support member 120 so as to be positioned adjacent to appendage 220, which is associated with limb 210. Lateral member 130 further includes secondary hinge area 134 and coupling member 132, wherein secondary hinge area 134 is moveably coupled to base member 131, and wherein coupling member 132 is coupled to secondary hinge area 134 such that coupling member 132 is configured to move relative to base member 131 so as to be positioned adjacent to support member 120. Furthermore, coupling member 132 is further configured to couple to support member 120 such that base member 131 is configured to support appendage 220 relative to limb 210.

Furthermore, an embodiment provides that support member 120 and base member 131 are both substantially rigid such that a degree of movement of appendage 220 relative to limb is hindered or precluded in response to appendage 220 being held firmly between support member 120 and base member 131. However, in so much as appendage 220 may be configured to move relative to limb 210 in various different directions, it is noted that the present technology is not limited to the diminishment or hindering of a specific type of movement. Indeed, appendage splint 110 may be implemented so as to hinder, diminish or preclude many different types of movements, such as, for example, a dorsiflexion, plantarflexion, abduction, adduction, inversion, eversion, supination and/or pronation of appendage 220 relative to limb 210.

Thus, as described above, an embodiment provides an appendage splint including a support member, and a lateral member coupled with a portion of the support member, wherein the lateral member is configured to move from a first position to a second position relative to the support member, and wherein the lateral member is further configured to couple with a different portion of the support member when the lateral member is positioned in the second position such that a degree of support is offered. For example, the lateral member may include a base member coupled with a portion of the support member, wherein the base member is configured to move from the first position to the second position. The lateral member may also include a coupling member coupled with the base member, wherein the coupling member is configured to couple with a different portion of the support member when the base member is positioned in the second position such that a degree of support is offered.

Moreover, as stated above, the lateral member is further configured to couple with a different portion of the support member when the lateral member is positioned in the second position such that a degree of support is offered. It is noted that this degree of support may be offered based on a degree of rigidity associated with the aforementioned base member. Furthermore, the support member may include a plurality of lateral extensions configured to flex so as to increase the degree of support, wherein the lateral member is coupled to a lateral extension from among the plurality of lateral extensions.

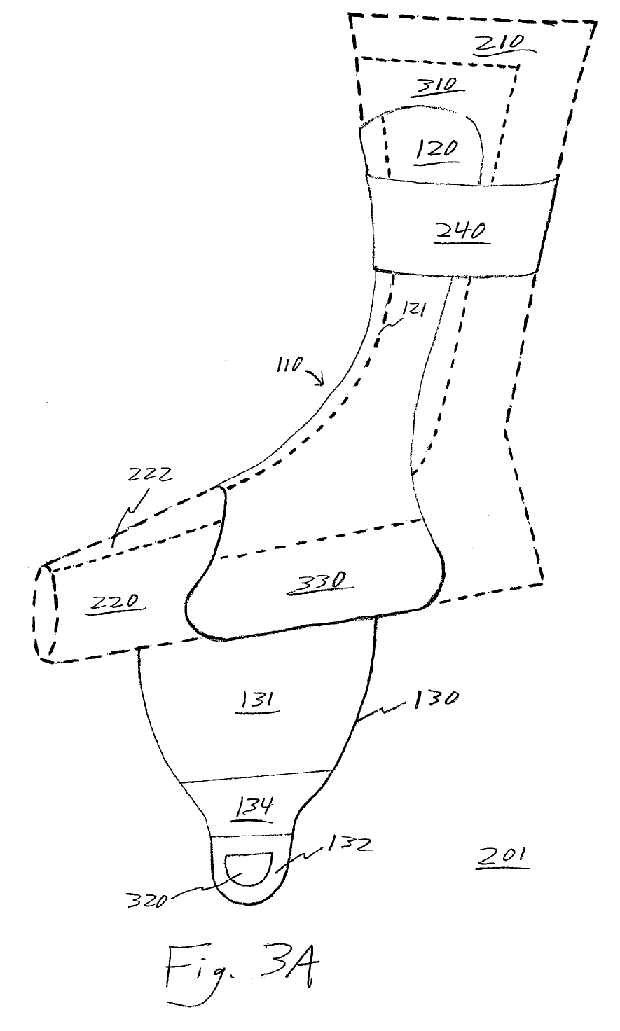

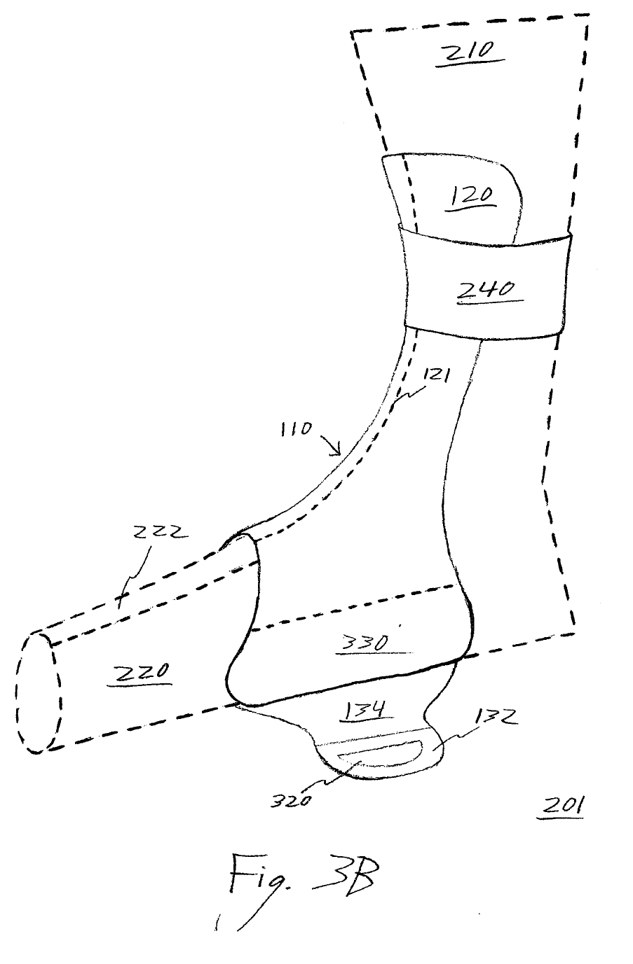

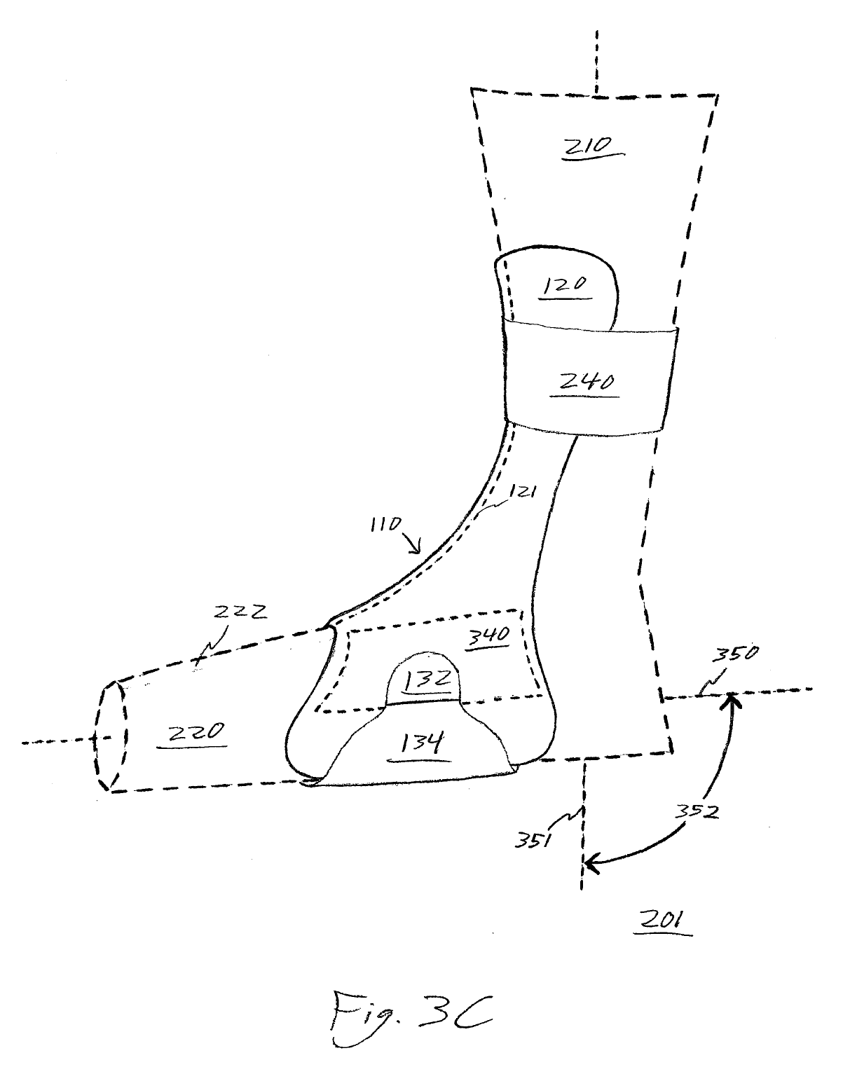

With reference now to FIGS. 3A, 3B and 3C, a second exemplary implementation 201 of appendage splint 110 in accordance with an embodiment is shown. Turning to FIG. 3A, it is noted that appendage splint 110 is shown to include support member 120, which is configured to be positioned adjacent to limb 210 such that major axis 121 of support member 120 extends along an anterior portion 310 of limb 210. Moreover, in an embodiment, appendage splint 110 is held against limb 210 such that support member 120 continues to contact or be positioned adjacent to anterior portion 310 during a movement of limb 210. To illustrate, an example provides that appendage splint 110 includes or is integrated with one or more wrapping elements, such as wrapping element 240 as described above, wherein the one or more wrapping elements are configured to wrap around limb 210 so as to hold support member 120 adjacent to anterior portion 310.

With reference still to FIG. 3A, in an embodiment, appendage splint 110 further includes lateral member 130, which is moveably coupled with support member 120. In particular, lateral member 130 includes base member 131, which is configured to move relative to support member 120 so as to be positioned adjacent to appendage 220, such as described above. For example, as shown in FIG. 3B, base member 131 has rotated relative to support member 120 so as to be positioned adjacent to a plantar portion (not shown) of appendage 220.

Furthermore, in one embodiment, appendage splint 110 includes coupling member 132, which is configured to couple with support member 120, such as shown in FIG. 3C, and such that base member 131 is configured to support appendage 220 relative to the limb 210. Consider the example wherein lateral member 130 is wrapped tightly around appendage 220, and wherein base member 131 and support member 120 are substantially rigid. In so much as lateral member 130 is wrapped tightly around appendage 220, appendage splint 110 is held firmly to appendage 220.

Moreover, in accordance with an exemplary implementation, in so much as base member 131 and support member 120 are both substantially rigid such that these portions of appendage splint 110 are not easily flexed in response to appendage 220 applying forces to these rigid portions, it is noted that a degree of movement of appendage 220 relative to limb 210 is precluded in response to appendage 220 being held firmly between support member 120 and base member 131, and support member 120 being held against limb 210, such as by wrapping element 240. In particular, it is noted that a degree of movement of appendage 220 toward limb 210 is hindered based on support member 120 being substantially rigid or resistive to flexing along major axis 121. It is further noted that a degree of movement of appendage 220 in a different direction, such as in a downward direction, relative to support member 120 is hindered based on base member 131 being substantially rigid or resistive to flexing along a portion of base member 131 that contacts a plantar portion of appendage 220.

Thus, an embodiment provides that support member 120 and base member 131 are both substantially rigid such that a degree of movement of appendage 220 relative to limb 210 is hindered or precluded in response to appendage 220 being held firmly between support member 120 and base member 131. However, in so much as appendage 220 may be configured to move relative to limb 210 in various different directions, it is noted that the present technology is not limited to the diminishment or hindering of a specific type of movement. Indeed, in an embodiment, a type of movement associated with such a hindered degree of movement is selected from among a group of movements consisting essentially of a dorsiflexion, plantarflexion, abduction, adduction, inversion, eversion, supination and pronation of appendage 220 relative to limb 210.

With reference still to FIG. 3A, in an embodiment, a contour of major axis 121 is configured to substantially conform to a shape of anterior portion 310 such that a surface of support member 120, such as a posterior surface of support member as shown in FIG. 1B, is configured to substantially engage a medial section of anterior portion 310 in response to support member 120 being positioned adjacent to limb 210 and major axis 121 extending along anterior portion 310. Indeed, in one exemplary implementation, support member 120 is positioned adjacent to limb 210 such that major axis 121 is substantially aligned with a medial section of anterior portion 310. Furthermore, a degree of movement of appendage 220 toward limb 210 is hindered based on support member 120 being substantially rigid or resistive to flexing along major axis 121, such as described above.

Moreover, and with reference still to major axis 121, as shown in FIGS. 3A-3C, in an embodiment, support member 120 is configured to be positioned adjacent to both limb 210 and appendage 220 such that major axis 121 extends along both anterior portion of limb 210 and dorsal portion 222 of appendage 220. Consider the example wherein anterior portion 310 of limb 210 is located adjacent to dorsal portion 222 of appendage 220. In so much as major axis 121 is configured to extend along both of these adjacent sections, an embodiment provides that support member 120 is configured to substantially conform to a shape of a joint region located between limb 210 and appendage 220.

Furthermore, in accordance with an embodiment, appendage splint 110 includes one or more flexible portions, such as optional flexible portion 330 shown in FIGS. 3A and 3B, wherein these one or more flexible portions are configured to flex toward appendage 220 such that the one or more flexible portions contact appendage 220, and such that an ability of base member 131 to support appendage 220 relative to limb 210 is increased. Indeed, pursuant to one exemplary configuration, support member 120 includes a plurality of lateral extensions, such as lateral extensions 124 shown in FIGS. 1A and 1B. These lateral extensions are substantially flexible such that the extensions are configured to conform to an outer surface of appendage 220, such as at generally opposite sides of appendage 220, when appendage 220 is positioned between these appendages. In this manner, an ability of appendage splint 110 to conform to a shape of appendage 220 is increased.

With reference now to FIG. 3C, an embodiment provides that coupling member 132 is configured to couple with support member 120 such that appendage 220 is held firmly between support member 120 and base member 131 based on an ability of appendage splint 210 to conform to a shape of appendage 220. Moreover, a degree of movement of appendage 220 relative to limb 210 is precluded based on a degree of rigidity associated with base member 131 and/or support member, such as described above.

To illustrate, an example provides that a base portion of support member 120 contacts dorsal portion 222 of appendage 220, as shown in FIG. 3C. Additionally, a plurality of lateral extensions, such as lateral extensions 124 shown in FIGS. 1A and 1B, conform to an outer surface of appendage 220, such as at generally opposite sides of appendage 220. Moreover, base member 131 is held taut against plantar portion 221 of appendage 220, as shown in FIG. 2C, in response to coupling member 132 being coupled with support member 120, as shown in FIG. 3C. Thus, due to an ability of appendage splint 110 to conform tightly around appendage 220, coupling member 132 provides a link that enables appendage splint 110 to hold appendage 220 firmly between the base of support member 120 and base member 131 such that a degree of movement of appendage 220 relative to limb 210 is diminished.

Thus, as described above, and with reference still to FIG. 3C, an embodiment provides that coupling member 132 is configured to couple with support member 120 such that base member 131 is configured to support appendage 220 relative to limb 210. Moreover, pursuant to one embodiment, support member 120 is further configured to support limb 210 relative to appendage 220 in response to coupling member 132 being coupled with support member 120. Consider the example where wrapping element 140, which is coupled with and/or wrapped around support member 120, is wrapped around limb 210 so as to hold support member 120 adjacent to limb 210, as shown in FIGS. 3A-3C, and where base member 131 is held taut against plantar portion 221 of appendage 220, as shown in FIG. 2C. In so much as support member 120 is substantially rigid along a medial section of support member, such as in a region of support member 120 along which major axis 121 extends, as shown in FIGS. 3A-3C, an ability of limb 210 to travel toward appendage 220 and/or an ability of appendage 220 to travel toward limb 210 is diminished.

With reference still to FIGS. 3A and 3B, an embodiment provides that coupling member 132 includes a coupler 320 configured to couple coupling member 132 to support member 120, such as shown in FIG. 3C, and such that base member 131 is configured to support appendage 220 relative to limb 210, as described above. To illustrate, coupler 320 may include an adhesive material that is configured to adhere to a portion of support member 120 with which coupler 320 comes into contact. Indeed, in one exemplary configuration, one or more hooks are coupled with or molded to a surface of appendage splint 110, wherein such hooks are configured to attach to a receptive coupling member that is also integrated with appendage splint 110.

Furthermore, in an embodiment, a snapping assembly is implemented that includes a plurality of snapping members that are configured to snap together such that the snapping members become coupled together. In particular, coupler 320 includes a first snapping member, while a second snapping member is coupled with or molded to a surface of support member 120. In this manner, coupler 320 may be moved adjacent to support member 120 such that the two snapping members may be snapped together such that coupled 320 becomes coupled to support member 120, and such that lateral member 130 becomes coupled with multiple portions of support member 120.

It is noted however, that various types of coupling devices may be implemented within the spirit and scope of the present technology. Accordingly, coupler 320 may be configured in accordance with various different types of coupling or adhesion technologies.

Additionally, in accordance with an embodiment, appendage splint 110 includes or is integrated with an outer layer that is coupled with, wrapped around or positioned adjacent to one or more portions of appendage splint 110, such as support member 120. Consider the example where at least a portion of an outer layer of material, such as exemplary portion 340 shown in FIG. 3C, is positioned adjacent to a portion of support member 120. Coupling member 132 is positioned adjacent to exemplary portion 340 of this outer layer, and is then attached to exemplary portion 340 such that support member 120, with which exemplary portion 340 is coupled, becomes coupled with coupling member 132. Indeed, pursuant to one embodiment, coupler 320 is configured to couple to at least a portion of the outer layer, such as exemplary portion 340, so as to couple coupling member 132 with support member 120, and such that base member 131 is configured to support appendage 220 relative to limb 210.

To further illustrate, an example provides that appendage splint 110 is wrapped in a fabric that substantially conforms to a shape of appendage splint 110. Coupler 320 is fastened to coupling member 132, as shown in FIGS. 3A and 3B, wherein coupler 320 includes a plurality of small hooks configured to grasp portions of the fabric. Coupling member 132 is then positioned adjacent to exemplary portion 340, as shown in FIG. 3C, and coupler 320 is coupled to fabric within exemplary portion 340 such that the aforementioned hooks grasp the fabric, and such that coupling member 132 becomes coupled with support member 120.

Thus, in an embodiment, coupling member 132 is configured to couple with support member 120 such that appendage 220 is supported relative to limb 210 based on a rigid nature of base member 131, as well as on a rigid nature of a medial portion of support member 120 that contacts both dorsal portion 222 of appendage 220 and anterior portion 310 of limb 210. With reference to FIG. 3C, it is noted that appendage 220 is supported relative to limb 210 such that a medial axis 350 of appendage 220 is positioned relative to a middle axis 351 of limb 210 as defined by a positioning angle 352. Although positioning angle 352 is not limited to any specific angular value, in one example, positioning angle 352 is approximately equal to 90 degrees, such that appendage 220 is held at approximately a 90 degree angle relative to limb 210.

For purposes of illustration, consider the example where limb 210 is a leg, and where appendage 220 is a foot that is moveably coupled with such leg about an ankle joint. Appendage splint 110 is configured to support the foot relative to the leg at a specific angle, such as to aid in the recovery of an adverse condition associated with the foot and/or ankle. In this manner, appendage splint 110 may be implemented so as to align certain body parts pursuant to an angle of alignment associated with appendage splint 110.

### Exemplary Arrangements

As described above, an appendage splint, in accordance with an embodiment, is disclosed herein, wherein the appendage splint is configured to support an appendage relative to a limb with which the appendage is moveably coupled such that a degree of movement of the appendage relative to the limb is hindered or precluded. Although various different configurations of an appendage splint may be implemented within the spirit and scope of the present technology, pursuant to one exemplary configuration, an appendage splint includes a support member that has a rigid section configured to be held adjacent to an anterior portion of a limb, wherein the rigid section of the support member is configured to offer a degree of support.

In an embodiment, the appendage splint further includes a lateral member that is moveably coupled with a first portion of the support member such that the lateral member is configured to wrap around an appendage associated with the aforementioned limb. It is noted, however, that various means may be implemented for coupling this lateral member with the support member. For example, the lateral member may be coupled with the support member by means of an external coupling element, such as a hinge element that is attached to both the lateral member and the support member, or by means of a sewn or stitched filament utilized to sew the lateral member to the support member. Indeed, in one exemplary arrangement, the lateral member is permanently coupled with or affixed to the support member, such as when both the lateral member and the support member are formed from the same piece of material.

Furthermore, the lateral member includes a coupling member configured to couple with a second portion of the support member when the lateral member is wrapped around the appendage, wherein the first and second portions of the support member may be contained within or associated with different lateral sides of the support member, such that the lateral member is coupled with different portions of the support member, and such that the appendage is supported relative to the aforementioned limb.

Additionally, an embodiment provides that the appendage splint includes a lateral extension located substantially opposite to the moveable lateral member such that an appendage may be positioned between the lateral extension and the lateral member when the appendage splint is held against or positioned adjacent to a limb. In addition, both the lateral extension and the lateral member conform to or wrap around the appendage when the aforementioned coupling member is coupled with the support member. This enables the appendage splint to substantially conform to a shape of the appendage that the appendage splint is to support relative to the leg with which the appendage is moveably coupled, and this degree of geometric conformation augments the ability of the appendage splint to hinder a degree of movement of the supported appendage relative to the aforementioned limb.

However, pursuant to one exemplary configuration, the lateral extension and the moveable lateral member do not couple together underneath the appendage, or adjacent to a plantar portion of such appendage. Indeed, in accordance with an embodiment, coupling these members together underneath such an appendage would create a structural weakness of the appendage splint adjacent to the plantar portion of the supported appendage, and this structural weakness might enable, for example, the appendage to push toward these coupled members and weaken or break the bond between such members, which would degrade the ability of the appendage splint to hinder a movement of the appendage relative to the limb.

Rather, an embodiment provides that both the lateral extension and the lateral member conform to or wrap around the appendage, yet the elongated lateral member is sufficiently long such that it is able to couple with a portion of the support member that is located away from a plantar surface of the appendage. Indeed, in accordance with one exemplary configuration, it is the lateral member, and not the lateral extension, that is positioned underneath or adjacent to the plantar portion of the appendage when the aforementioned coupling member is coupled with the support member. Instead, the lateral extension is positioned adjacent to a lateral surface of the appendage, wherein this lateral surface is located between the dorsal and plantar surfaces of the appendage within an outer surface area of such appendage.

Furthermore, in an embodiment, the lateral member includes a base member that is substantially rigid, and it is this rigid base member that is positioned underneath or adjacent to a plantar portion of an appendage when the lateral member is wrapped around the appendage. Due to a degree of rigidity of this base member, as well as to an ability of the lateral member to be wrapped taut around the appendage, a degree of movement of such appendage relative to a limb with which the appendage is moveably coupled is hindered to a greater degree than would be achievable if the portion of the lateral member contacting the plantar portion of the appendage were configured to be substantially flexible or non-rigid.

Thus, in contrast to a clam shell design that includes two moveable or flexible members that are configured to wrap around an appendage and couple or clasp together underneath the appendage, an embodiment provides an appendage splint that includes a base member that is moveably coupled with an upper support member by means of a hinge element. In addition, a coupling element, which may include a rigid tab, is also moveably coupled with the moveable base member such that the coupling element moves adjacent to the support member, and couples with a portion of the support member such that the rigid base member is held underneath or adjacent to a plantar surface of the appendage. However, it is noted that the rigid support member, and not the coupling member, is positioned underneath the appendage when the coupling member has been coupled with the upper support member.

The foregoing notwithstanding, pursuant to one example, an appendage splint as discussed herein is easier to mount to an appendage than a splint having a clam shell design, such as described above. Consider the example where a clam shell design includes two lateral straps each coupled with a different side of a support structure, wherein these two lateral straps are each tightly wrapped around an appendage and then coupled together, such as adjacent to a plantar portion of the appendage. In so much as a degree of structural weakness is associated with these two lateral straps being coupled together, such as described above, a third strap or wrapping member may be wrapped around the coupled lateral straps so as to provide further structural support. Thus, an example provides that a clam shell design utilizes at least three straps to effectively mount a splint to an appendage.

In contrast, an embodiment provides an appendage splint that is distinguishable from such a clam shell design. In particular, an appendage splint is provided that includes a single lateral member coupled with a support member, wherein the lateral member is configured to wrap substantially around an appendage and couple with a second portion of the appendage. In so much as a rigid nature of the support structure hinders an ability of the appendage to move toward the limb with which the appendage is moveably coupled, and in so much as the lateral member is not coupled with the support structure beneath a plantar surface of the appendage, the application of a force by the appendage to the appendage splint will not easily sever a bond between the lateral member and the rigid support structure.

Thus, an example provides that a single, elongated lateral member or strap may be implemented so as to achieve a degree of structural integrity that a clam shell design, such as described above, would utilize at least three straps to achieve. In this manner, it is noted that an appendage splint as discussed herein may be more easily mounted to an appendage in comparison to the aforementioned clam shell design.

Moreover, this appendage splint may also be easier to adjust, adapt to, and remove from an appendage based on the use of a single lateral strap. Consider the example where a splint as described herein includes a single strap, along with a hood or loop fastener associated therewith, wherein the single strap is configured to support and stabilize an ankle joint or area. Due to a degree of geometric conformity associated with the appendage splint, as well as the ease of operation associated with the single strap, the appendage splint may be relatively easily adapted to, and subsequently removed from, for example, a lower ankle.

Furthermore, in so much as multiple lateral straps are not utilized, an embodiment provides that an appendage splint as described herein is more comfortable to wear than a splint having a clam shell design. Consider the example where each implemented strap in a clam shell design is wrapped tightly around an appendage such that each strap contributes to a level of discomfort associated with the splint. By minimizing the number of straps that are to be implemented, an embodiment as described herein may be implemented so as to optimize a level of comfort associated with an appendage splint.

The foregoing notwithstanding, in an embodiment, an appendage splint includes a splint member having varying degrees of thickness, wherein this splint member is divided into multiple sections based on these varying degrees of thickness. In particular, these different sections may include a support member, a base member, a coupling member, and a plurality of hinge elements, such as described above, wherein different degrees of thickness are associated with different elements of the splint member.

With reference still to the previous embodiment, it is noted that this configuration of a splint member is not obvious in view of prior splint devices at least because the differing degrees of material thickness, flexibility and rigidity associated with the different elements of the aforementioned splint member are difficult to achieve during an injection molding process implemented to fabricate such a splint member. Therefore, in accordance with an exemplary scenario, the fabrication of such a splint member may be more difficult than conventional designs, and may even be more costly. Accordingly, it is noted that a person having ordinary skill in the art would be motivated to not design or fabricate a splint member such as described herein due to the relative difficulty and expense associated therewith.

Although various configurations and implementations are discussed herein, these configurations and implementations are presented for purposes of illustration, and are not intended to suggest any limitation as to the scope of use or functionality of the present technology. Moreover, the present technology is not to be interpreted as having any dependency or relation to any one or combination of components illustrated in the disclosed examples.

Additionally, it is noted that various exemplary implementations discussed herein describe the use of appendage splint 110 so as to support appendage 220 relative to limb 210. It is noted that limb 210 and appendage 220 may correspond to a limb and appendage, respectively, of a living being, such as a human or animal, or such as a plant (e.g., a tree having a trunk and a plurality of branches extending therefrom). However, the spirit and scope of the present technology is not limited to the implementation of an appendage splint with limbs and appendages corresponding to living beings. Indeed, the present technology may also be implemented so as to support an appendage relative to structure with which the appendage is coupled, wherein the appendage and structure do not correspond to body parts of a living being.

### Exemplary Method of Use

In accordance with an embodiment, an appendage splint, which may be utilized as a dorsal night splint, is configured to enable a stabilization of an ankle in a neutral position, such as to treat severe ankle sprains, Achilles tendonitis/repairs, foot drop and plantar fasciitis/heel pain. For example, the appendage splint is configured to offer a patient strong, rigid foot support that effectively holds the foot in a neutral position during rest, which helps alleviate the discomfort caused by plantar fasciitis or other foot-related ailments. Moreover, the splint is configured to be universal, in that it may fit either the right or left foot, and may be customized to fit patient's of different size. Furthermore, the splint is less bulky than conventional designs, thereby promoting a higher degree of patient compliance.

To illustrate an exemplary method of use, an embodiment provides that the appendage splint is configured to be fitted as follows: While sitting, the patient places the appendage splint on a flat surface, and un-hooks or detaches the coupling member tab from a side of the brace. The patient next places his/her foot within the splint such that the patient's calf is pressed against the brace, and then pulls and securely attaches the coupled member tab such that the brace is fit snug around the patient's foot. Additionally, the patient applies a wrapping element (e.g., a calf strap) around the patient's leg so as to secure the brace to the leg. Once properly fitted, the splint secures to the top of the patient's foot such that the foot is held relative to the patient's leg at an angle of approximately 90°, such as when the patient is asleep.

The foregoing notwithstanding, in contrast to past support splints, an embodiment provides a low profile design that leaves more of the foot and leg exposed for a cooler, more comfortable wear. For example, the splint may include an inner shell surrounded by a soft memory foam liner configured to provide increased comfort. Moreover, the adjustable tab of the coupling member and the strong stretch strap of the wrapping element are configured to function in tandem so as to provide a custom fit with the leg and foot. Furthermore, an exemplary implementation provides that the wrapping element may be easily cleaned or washed (e.g., using cold water and mild soap), such as by hand.

### SUMMARY CONCEPTS

It is noted that the foregoing discussion has presented at least the following concepts:
Concept 0. An appendage splint including or comprising:

a support member; and

a lateral member moveably coupled with a portion of the support member, the lateral member being configured to wrap so as to couple with another portion of the support member.
Concept 1. An appendage splint including or comprising:

a support member configured to be positioned adjacent to a limb such that a major axis of the support member extends along an anterior portion of the limb;

a base member associated with the support member, the base member configured to move relative to the support member so as to be positioned adjacent to an appendage associated with the limb; and

a coupling member associated with the base member, the coupling member configured to couple with the support member such that the base member is configured to support the appendage relative to the limb.
Concept 2. The appendage splint of Concept 1, wherein a contour of the major axis is configured to substantially conform to a shape of the anterior portion such that a surface of the support member is configured to substantially engage a medial section of the anterior portion in response to the support member being positioned adjacent to the limb and the major axis extending along the anterior portion.
Concept 3. The appendage splint of Concept 1, wherein the support member is further configured to support the limb relative to the appendage in response to the coupling member being coupled with the support member.
Concept 4. The appendage splint of Concept 3, further including or comprising:

a wrapping element associated with the support member, the wrapping element configured to wrap around the limb so as to hold the support member adjacent to the limb, and so as to enable the support member to support the limb relative to the appendage.
Concept 5. The appendage splint of Concept 3, further including or comprising:

a force absorbing element associated with the support member, the force absorbing element configured to absorb a force associated with the support member being held adjacent to the limb such that a level of comfort associated with the support member being held adjacent to the limb is increased.
Concept 6. The appendage splint of Concept 1, wherein the support member includes or comprises:

a plurality of flexible portions configured to flex toward the appendage such that the plurality of flexible portions contact the appendage, and such that an ability of the base member to support the appendage relative to the limb is increased.
Concept 7. The appendage splint of Concept 1, further including or comprising:

a primary hinge area coupled to the support member and the base member, the primary hinge area configured to enable the base member to move relative to the support member; and

a secondary hinge area coupled to the base member and the coupling member, the secondary hinge area configured to enable the coupling member to move relative to the base member.
Concept 8. The appendage splint of Concept 7, wherein the support member, the primary hinge area, the base member, the secondary hinge area, and the coupling member include or comprise different sections of a single splint member having varying degrees of thickness, and wherein different degrees of thickness are associated with the different sections.
Concept 9. The appendage splint of Concept 7, wherein degrees of rigidity associated with the support member and the base member are greater than different degrees of rigidity associated with the primary hinge area and the secondary hinge area.
Concept 10. The appendage splint of Concept 9, wherein the coupling member is further configured to couple with the support member such that the appendage is held between the support member and the base member, and such that a degree of movement of the appendage relative to the limb is precluded based on a degree of rigidity associated with the base member.
Concept 11. An appendage splint including or comprising:

a support member configured to be positioned adjacent to a limb; and

a lateral member moveably coupled with a portion of the support member, the lateral member including or comprising:

a base member configured to move relative to the support member so as to be positioned adjacent to a plantar portion of an appendage associated with the limb; and

a coupling member associated with the base member, the coupling member configured to couple the base member with a different portion of the support member such that the base member is configured to support the appendage relative to the limb.
Concept 12. The appendage splint of Concept 11, wherein the support member is further configured to be positioned adjacent to the limb and the appendage such that a major axis of the support member extends along an anterior portion of the limb and a dorsal portion of the appendage
Concept 13. The appendage splint of Concept 12, wherein the anterior portion of the limb is located adjacent to the dorsal portion of the appendage.
Concept 14. The appendage splint of Concept 12, wherein the plantar portion of the appendage is located substantially opposite to the dorsal portion of the appendage along a length of the appendage.
Concept 15. The appendage splint of Concept 12, wherein the support member includes or comprises generally opposite sides divided by the major axis, the portion of the support member is located within a first side from among the generally opposite sides, and the different portion of the support member is located within a different side from among the generally opposite sides.
Concept 16. The appendage splint of Concept 11, wherein the different portion of the support member is not located adjacent to the plantar portion of the appendage.
Concept 17. The appendage splint of Concept 11, wherein the base member is substantially rigid such that a degree of movement of the appendage relative to the limb is precluded in response to the appendage being held between the support member and the base member.
Concept 18. The appendage splint of Concept 17, wherein a type of movement associated with the degree of movement is selected from among a group of movements consisting essentially of a dorsiflexion, plantarflexion, abduction, adduction, inversion, eversion, supination and pronation of the appendage relative to the limb.
Concept 19. An appendage splint including or comprising:

a support member configured to be positioned adjacent to an anterior portion of a limb; and

a lateral member associated with the support member, the lateral member including or comprising:

a primary hinge area moveably coupled with the support member;

a base member coupled with the primary hinge area such that the base member is configured to move relative to the support member so as to be positioned adjacent to an appendage associated with the limb;

a secondary hinge area moveably coupled with the base member; and

a coupling member coupled with the secondary hinge area such that the coupling member is configured to move relative to the base member so as to be positioned adjacent to the support member, and the coupling member being further configured to couple with the support member such that the base member is configured to support the appendage relative to the limb.
Concept 20. The appendage splint of Concept 19, wherein the support member is further configured to support the limb relative to the appendage in response to the coupling member being coupled with the support member.
Concept 21. The appendage splint of Concept 20, further including or comprising:

a wrapping element associated with the support member, the wrapping element configured to wrap around the limb so as to hold the support member adjacent to the limb, and so as to enable the support member to support the limb relative to the appendage.
Concept 22. The appendage splint of Concept 19, wherein the support member includes or comprises:

a plurality of flexible portions configured to flex toward the appendage such that the plurality of flexible portions contact the appendage, and such that an ability of the base member to support the appendage relative to the limb is increased.
Concept 23. The appendage splint of Concept 19, wherein the coupling member includes or comprises:

a coupler configured to couple the coupling member to the support member such that the base member is configured to support the appendage relative to the limb.
Concept 24. The appendage splint of Concept 19, wherein the appendage splint further includes or comprises:

an outer layer associated with the support member, at least a portion of the outer layer being positioned adjacent to a portion of the support member.
Concept 25. The appendage splint of Concept 24, wherein the coupling member includes or comprises:

a coupler configured to couple to the at least a portion of the outer layer so as to couple the coupling member with the support member such that the base member is configured to support the appendage relative to the limb.
Concept 26. An appendage splint including or comprising:

a support member configured to be positioned adjacent to a limb such that a major axis of the support member extends along an anterior portion of the limb;

a primary hinge area moveably coupled to the support member;

a base member coupled to the primary hinge area such that the base member is configured to move relative to the support member so as to be positioned adjacent to an appendage associated with the limb;

a secondary hinge area moveably coupled to the base member; and

a coupling member coupled to the secondary hinge area such that the coupling member is configured to move relative to the base member so as to be positioned adjacent to the support member, and the coupling member being further configured to couple with the support member such that the base member is configured to support the appendage relative to the limb.
Concept 27. The appendage splint of Concept 26, wherein the support member is further configured to support the limb relative to the appendage in response to the coupling member being coupled to the support member.
Concept 28. The appendage splint of Concept 27, further including or comprising:

a wrapping element associated with the support member, the wrapping element configured to wrap around the limb so as to hold the support member adjacent to the limb, and so as to enable the support member to support the limb relative to the appendage.
Concept 29. The appendage splint of Concept 26, wherein the support member includes or comprises:

a plurality of flexible portions configured to flex toward the appendage such that the plurality of flexible portions contact the appendage, and such that an ability of the base member to support the appendage relative to the limb is increased.
Concept 30. The appendage splint of Concept 26, wherein the coupling member includes or comprises:

a coupler configured to couple the coupling member to the support member such that the base member is configured to support the appendage relative to the limb.
Concept 31. The appendage splint of Concept 26, wherein the appendage splint further includes or comprises:

an outer layer associated with the support member, at least a portion of the outer layer being positioned adjacent to a portion of the support member.
Concept 32. The appendage splint of Concept 31, wherein the coupling member includes or comprises:

a coupler configured to couple to the at least a portion of the outer layer so as to couple the coupling member with the support member such that the base member is configured to support the appendage relative to the limb.
Concept 33. The appendage splint of Concept 26, wherein the support member, the primary hinge area, the base member, the secondary hinge area, and the coupling member include or comprise different sections of a single splint member having varying degrees of thickness, and wherein different degrees of thickness are associated with the different sections.
Concept 34. The appendage splint of Concept 33, wherein degrees of thickness associated with the support member and the base member are greater than different degrees of thickness associated with the primary hinge area and the secondary hinge area such that the support member and the base member are substantially rigid in comparison to the primary hinge area and the secondary hinge area, and such that the primary hinge area and the secondary hinge area are substantially flexible in comparison to the support member and the base member.
Concept 35. The appendage splint of Concept 34, wherein the primary hinge area is configured to enable the base member to move relative to the support member based on a degree of flexibility associated with the primary hinge area being greater than another degree of flexibility associated with the support member.
Concept 36. The appendage splint of Concept 34, wherein the secondary hinge area is configured to enable the coupling member to move relative to the base member based on a degree of flexibility associated with the secondary hinge area being greater than another degree of flexibility associated with the base member.
Concept 37. An appendage splint including or comprising:

a support member; and

a lateral member coupled with a portion of the support member, the lateral member configured to move from a first position to a second position relative to the support member, and the lateral member further configured to couple with a different portion of the support member when the lateral member is positioned in the second position such that a degree of support is offered.
Concept 38. The appendage splint of Concept 37, wherein the lateral member includes or comprises:

a base member coupled with the portion of the support member, the base member configured to move from the first position to the second position; and

a coupling member coupled with the base member, the coupling member configured to couple with the different portion of the support member when the base member is positioned in the second position such that the degree of support is offered.
Concept 39. The appendage splint of Concept 38, wherein the degree of support is offered based on a degree of rigidity associated with the base member.
Concept 40. The appendage splint of Concept 38, further including or comprising:

a primary hinge area coupled to the support member and the base member, the primary hinge area configured to enable the base member to move relative to the support member; and

a secondary hinge area coupled to the base member and the coupling member, the secondary hinge area configured to enable the coupling member to move relative to the base member.
Concept 41. The appendage splint of Concept 37, wherein the support member includes or comprises:

a plurality of lateral extensions configured to flex so as to increase the degree of support.
Concept 42. The appendage splint of Concept 41, wherein the lateral member is coupled to a lateral extension from among the plurality of lateral extensions.
Concept 43. An appendage splint including or comprising:

a support member;

a base member coupled with a portion of the support member, the base member configured to move from a first position to a second position relative to the support member; and

a coupling member coupled with the base member, the coupling member configured to couple with a different portion of the support member when the base member is positioned in the second position such that a degree of support is offered.
Concept 44. The appendage splint of Concept 43, wherein the degree of support is offered based on a degree of rigidity associated with the base member.
Concept 45. The appendage splint of Concept 43, wherein a degree of movement is hindered in response to the degree of support being utilized.
Concept 46. The appendage splint of Concept 43, wherein the support member includes or comprises:

a plurality of lateral extensions configured to flex so as to increase the degree of support.
Concept 47. The appendage splint of Concept 43, further including or comprising:

a wrapping element associated with the support member, the wrapping element configured to hold the support member in place so as to increase the degree of support.
Concept 48. The appendage splint of Concept 47, further including or comprising:

a force absorbing element associated with the support member, the force absorbing element configured to absorb a force associated with the support member being held in place such that a level of comfort associated with the support member being held in place is increased.
Concept 49. The appendage splint of Concept 43, further including or comprising:

a primary hinge area coupled to the support member and the base member, the primary hinge area configured to enable the base member to move relative to the support member; and

a secondary hinge area coupled to the base member and the coupling member, the secondary hinge area configured to enable the coupling member to move relative to the base member.
Concept 50. The appendage splint of Concept 49, wherein the support member, the primary hinge area, the base member, the secondary hinge area, and the coupling member include or comprise different sections of a single splint member having varying degrees of thickness, and wherein different degrees of thickness are associated with the different sections.
Concept 51. The appendage splint of Concept 49, wherein degrees of rigidity associated with the support member and the base member are greater than different degrees of rigidity associated with the primary hinge area and the secondary hinge area.
Concept 52. The appendage splint of Concept 43, wherein the coupling member includes or comprises:

a coupler configured to couple the coupling member to the support member such that the degree of support is offered.
Concept 53. The appendage splint of Concept 43, wherein the appendage splint further includes or comprises:

an outer layer associated with the support member, at least a portion of the outer layer being positioned adjacent to the different portion of the support member.
Concept 54. The appendage splint of Concept 53, wherein the coupling member includes or comprises:

a coupler configured to couple to the at least a portion of the outer layer so as to couple the coupling member with the support member such that the degree of support is offered.
Concept 55. An appendage splint including or comprising:

a support member including or comprising a lateral extension; and

a lateral member associated with the support member such that a first portion of the lateral member is coupled with the lateral extension, the lateral member configured to move relative to the support member such that a second portion of the lateral member is able to couple with the support member, and such that a degree of support is offered in response to the second portion of the lateral member being coupled with the support member.
Concept 56. The appendage splint of Concept 55, wherein the lateral member includes or comprises:

a base member coupled with the support member, wherein the degree of support is offered based on a degree of rigidity associated with the base member.
Concept 57. The appendage splint of Concept 55, wherein the lateral extension is configured to flex so as to increase the degree of support.
Concept 58. An appendage splint including or comprising:

a support member including or comprising a lateral member configured to wrap and couple with a portion of the support member.
Concept 59. The appendage splint of Concept 58, wherein the portion of the support member is included within or located adjacent to a dorsal section of the support member.
Concept 60. An appendage splint including or comprising:

a support member including or comprising a lateral member coupled with a portion of the support member, the lateral member configured to move from a first position to a second position relative to the portion, and the lateral member further configured to couple with a different portion of the support member when the lateral member is positioned in the second position.
Concept 61. The appendage splint of Concept 60, wherein the portion and the different portion of the support member are included within a dorsal section of the support member such that the lateral member is configured to couple with at least two different portions of the dorsal section.
Concept 62. The appendage splint of Concept 60, wherein the lateral member includes or comprises:

a base member coupled with the portion of the support member, the base member configured to move from the first position to the second position.
Concept 63. The appendage splint of Concept 62, further including or comprising:

a primary hinge area coupled between the support member and the base member, the primary hinge area configured to enable the base member to move relative to the support member.
Concept 64. The appendage splint of Concept 62, wherein the lateral member further includes or comprises:

a coupling member coupled with the base member, the coupling member configured to couple with the different portion of the support member when the base member is positioned in the second position.
Concept 65. The appendage splint of Concept 64, wherein the lateral member further includes or comprises:

a secondary hinge area coupled between the base member and the coupling member, the secondary hinge area configured to enable the coupling member to move relative to the base member.
Concept 66. The appendage splint of Concept 60, wherein the support member includes or comprises:

a plurality of flexible lateral extensions, wherein the lateral member is coupled with a lateral extension from among the plurality of lateral extensions.
Concept 67. An appendage splint including or comprising:

a support member; and

a lateral member coupled with a portion of the support member, the lateral member configured to move from a first position to a second position relative to the support member, and the lateral member further configured to couple with a different portion of the support member when the lateral member is positioned in the second position.
Concept 68. The appendage splint of Concept 67, wherein the lateral member includes or comprises:

a base member coupled with the portion of the support member, the base member configured to move from the first position to the second position; and

a coupling member coupled with the base member, the coupling member configured to couple with the different portion of the support member when the base member is positioned in the second position.
Concept 69. The appendage splint of Concept 68, wherein a degree of support is offered based on a degree of rigidity of the base member.
Concept 70. The appendage splint of Concept 68, wherein the coupling member includes or comprises:

a coupler configured to couple the coupling member to the support member.
Concept 71. The appendage splint of Concept 68, further including or comprising:

a primary hinge area coupled with the support member and the base member, the primary hinge area configured to enable the base member to move relative to the support member; and

a secondary hinge area coupled with the base member and the coupling member, the secondary hinge area configured to enable the coupling member to move relative to the base member.
Concept 72. The appendage splint of Concept 71, wherein the support member, the primary hinge area, the base member, the secondary hinge area, and the coupling member comprise different sections of a single splint member having varying degrees of thickness, and wherein different degrees of thickness are associated with the different sections.
Concept 73. The appendage splint of Concept 72, wherein degrees of rigidity associated with the support member and the base member are greater than different degrees of rigidity associated with the primary hinge area and the secondary hinge area.
Concept 74. The appendage splint of Concept 67, wherein the appendage splint further includes or comprises:

an outer layer associated with the support member, at least a portion of the outer layer being positioned adjacent to the different portion of the support member.
Concept 75. The appendage splint of Concept 74, wherein the coupling member includes or comprises:

a coupler configured to couple to the at least a portion of the outer layer so as to couple the coupling member with the support member.
Concept 76. The appendage splint of Concept 67, wherein the support member includes or comprises:

a plurality of flexible lateral extensions, wherein the lateral member is coupled with a lateral extension from among the plurality of lateral extensions.
Concept 77. The appendage splint of Concept 67, further including or comprising:

a wrapping element associated with the support member, the wrapping element configured to wrap and hold the support member in place.
Concept 78. The appendage splint of Concept 77, further including or comprising:

a force absorbing element associated with the support member, the force absorbing element configured to absorb a force when the support member is held in place.
Concept 79. An appendage splint including or comprising:

a support member including or comprising a lateral member configured to wrap and couple with a portion of the support member.
Concept 80. The appendage splint of Concept 79, wherein the portion of the support member is included within or located adjacent to a dorsal section of the support member.
Concept 81. The appendage splint of Concept 79, wherein the portion of the support member is included within or located adjacent to a lateral section of the support member.
Concept 82. The appendage splint of Concept 79, wherein the support member is configured to be positioned adjacent to a limb such that a major axis of the support member extends along an anterior portion of the limb.
Concept 83. The appendage splint of Concept 79, wherein the lateral member includes or comprises:

a base member configured to be positioned adjacent to a plantar portion of an appendage; and

a coupling member extending from the base member, the coupling member configured to be displaced around or adjacent to a dorsal section of the support member.
Concept 84. The appendage splint of Concept 83, wherein the coupling member is further configured to be displaced adjacent to a plantar surface of the base member and couple with the plantar surface.
Concept 85. An appendage splint including or comprising:

a support member including or comprising a lateral member, the lateral member configured to move from a first position to a second position relative to the support member and couple with the support member when the lateral member is positioned in the second position.
Concept 86. The appendage splint of Concept 85, wherein the lateral member includes or comprises:

a base member coupled with the portion of the support member, the base member configured to move from the first position to the second position.
Concept 87. The appendage splint of Concept 86, further including or comprising:

a primary hinge area coupled between the support member and the base member, the primary hinge area configured to enable the base member to move relative to the support member.
Concept 88. The appendage splint of Concept 86, wherein the lateral member further includes or comprises:

a coupling member coupled with the base member, the coupling member configured to couple with the different portion of the support member when the base member is positioned in the second position.
Concept 89. The appendage splint of Concept 88, wherein the lateral member further includes or comprises:

a secondary hinge area coupled between the base member and the coupling member, the secondary hinge area configured to enable the coupling member to move relative to the base member.
Concept 90. The appendage splint of Concept 85, wherein the support member includes or comprises:

a plurality of flexible lateral extensions, wherein the lateral member is coupled with a lateral extension from among the plurality of flexible lateral extensions.
Concept 91. An appendage splint including or comprising:

a support member including or comprising a lateral extension extending from the support member; and

a lateral member extending from the lateral extension, the lateral member including or comprising a base member configured to move from a first position to a second position relative to the support member, and the lateral member further including or comprising a connecting member extending from the base member, the connecting member configured to connect with the support member when the base member is positioned in the second position.
Concept 92. The appendage splint of Concept 91, wherein the base member is rigid, and wherein the connecting member is a coupling member.
Concept 93. The appendage splint of Concept 92, wherein the base member is configured to offer a degree of support based on a degree of rigidity of the base member in response to the connecting member being coupled with the support member.
Concept 94. The appendage splint of Concept 91, wherein the connecting member includes or comprises:

a coupler configured to attach the connecting member to the support member when the base member is positioned in the second position.
Concept 95. The appendage splint of Concept 91, further including or comprising:

a primary hinge area positioned between the lateral extension and the base member, the primary hinge area configured to enable the base member to move relative to the support member; and

a secondary hinge area positioned between the base member and the connecting member, the secondary hinge area configured to enable the connecting member to move relative to the base member.
Concept 96. The appendage splint of Concept 95, wherein the support member, the primary hinge area, the base member, the secondary hinge area, and the connecting member comprise different sections of a single splint member having varying degrees of thickness, and wherein different degrees of thickness are associated with the different sections.
Concept 97. The appendage splint of Concept 96, wherein degrees of rigidity associated with the support member and the base member are greater than other degrees of rigidity associated with the primary hinge area and the secondary hinge area.
Concept 98. The appendage splint of Concept 91, wherein the appendage splint further includes or comprises:

an outer layer associated with the support member, at least a portion of the outer layer being positioned adjacent to a portion of the support member with which the connecting member is configured to coupled when the base member is positioned in the second position.
Concept 99. The appendage splint of Concept 98, wherein the connecting member includes or comprises:

a coupler configured to couple to the at least a portion of the outer layer so as to couple the connecting member with the support member.
Concept 100. The appendage splint of Concept 91, further including or comprising:

a wrapping element associated with the support member, the wrapping element configured to wrap and hold the support member in place.
Concept 101. The appendage splint of Concept 100, further including or comprising:

a force absorbing element associated with the support member, the force absorbing element configured to absorb a force when the support member is held in place.
Concept 102. An appendage splint including or comprising:

a support member including or comprising a lateral extension extending from the support member; and

a lateral member extending from the lateral extension, the lateral member including or comprising a rigid base member configured to move from a first position to a second position relative to the support member, and the lateral member further including or comprising a coupling member extending from the rigid base member, the coupling member configured to couple with the support member when the rigid base member is positioned in the second position.

Although various exemplary embodiments of the present technology are described herein in a language specific to structural features and/or methodological acts, the subject matter defined in the appended claims is not necessarily limited to the specific features or acts described above. Rather, the specific features and acts described above are disclosed as exemplary forms of implementing the claims.

What is claimed is:

1. An appendage splint comprising:
a support member including a lateral member configured to wrap and couple with a portion of said support member.

2. The appendage splint of claim 1, wherein said portion of said support member is included within or located adjacent to a dorsal section of said support member.

3. The appendage splint of claim 1, wherein said portion of said support member is included within or located adjacent to a lateral section of said support member.

4. The appendage splint of claim 1, wherein said support member is configured to be positioned adjacent to a limb such that a major axis of said support member extends along an anterior portion of said limb.

5. The appendage splint of claim 1, wherein said lateral member comprises:
a base member configured to be positioned adjacent to a plantar portion of an appendage; and
a coupling member extending from said base member, said coupling member configured to be displaced around or adjacent to a dorsal section of said support member.

6. The appendage splint of claim 5, wherein said coupling member is further configured to be displaced adjacent to a plantar surface of said base member and couple with said plantar surface.

7. An appendage splint comprising:
a support member including a lateral member, said lateral member configured to move from a first position to a second position relative to said support member and couple with said support member when said lateral member is positioned in said second position.

8. The appendage splint of claim 7, wherein said lateral member comprises:
a base member coupled with said portion of said support member, said base member configured to move from said first position to said second position.

9. The appendage splint of claim 8, further comprising:
a primary hinge area coupled between said support member and said base member, said primary hinge area configured to enable said base member to move relative to said support member.

10. The appendage splint of claim 8, wherein said lateral member further comprises:
a coupling member coupled with said base member, said coupling member configured to couple with said different portion of said support member when said base member is positioned in said second position.

11. The appendage splint of claim 10, wherein said lateral member further comprises:
a secondary hinge area coupled between said base member and said coupling member, said secondary hinge area configured to enable said coupling member to move relative to said base member.

12. The appendage splint of claim 7, wherein said support member comprises:
a plurality of flexible lateral extensions, wherein said lateral member is coupled with a lateral extension from among said plurality of flexible lateral extensions.

13. An appendage splint comprising:
a support member including a lateral extension extending from said support member; and
a lateral member extending from said lateral extension, said lateral member including a base member configured to move from a first position to a second position relative to said support member, and said lateral member further including a connecting member extending from said base member, said connecting member configured to connect with said support member when said base member is positioned in said second position.

14. The appendage splint of claim 13, wherein said base member is rigid, and wherein said connecting member is a coupling member.

15. The appendage splint of claim 14, wherein said base member is configured to offer a degree of support based on a degree of rigidity of said base member in response to said connecting member being coupled with said support member.

16. The appendage splint of claim 13, wherein said connecting member comprises:
a coupler configured to attach said connecting member to said support member when said base member is positioned in said second position.

17. The appendage splint of claim 13, further comprising:
a primary hinge area positioned between said lateral extension and said base member, said primary hinge area configured to enable said base member to move relative to said support member; and
a secondary hinge area positioned between said base member and said connecting member, said secondary hinge area configured to enable said connecting member to move relative to said base member.

18. The appendage splint of claim 17, wherein said support member, said primary hinge area, said base member, said secondary hinge area, and said connecting member comprise different sections of a single splint member having varying degrees of thickness, and wherein different degrees of thickness are associated with said different sections.

19. The appendage splint of claim 18, wherein degrees of rigidity associated with said support member and said base member are greater than other degrees of rigidity associated with said primary hinge area and said secondary hinge area.

20. The appendage splint of claim 13, wherein said appendage splint further comprises:
an outer layer associated with said support member, at least a portion of said outer layer being positioned adjacent to a portion of said support member with which said connecting member is configured to coupled when said base member is positioned in said second position.

21. The appendage splint of claim 20, wherein said connecting member comprises:
a coupler configured to couple to said at least a portion of said outer layer so as to couple said connecting member with said support member.

22. The appendage splint of claim 13, further comprising:
a wrapping element associated with said support member, said wrapping element configured to wrap and hold said support member in place.

23. The appendage splint of claim 22, further comprising:
a force absorbing element associated with said support member, said force absorbing element configured to absorb a force when said support member is held in place.

24. An appendage splint comprising:
a support member including a lateral extension extending from said support member; and
a lateral member extending from said lateral extension, said lateral member including a rigid base member configured to move from a first position to a second position relative to said support member, and said lateral member further including a coupling member extending from said rigid base member, said coupling member configured to couple with said support member when said rigid base member is positioned in said second position.
